(19) United States
(12) Patent Application Publication
(10) Pub. No.: US 2024/0413352 A1
(43) Pub. Date: Dec. 12, 2024

(54) ENZYME ELECTRODE HAVING AN INTEGRATED RESERVOIR

(71) Applicants: CENTRE NATIONAL DE LA RECHERCHE SCIENTIFIQUE, Paris (FR); UNIVERSITÉ GRENOBLE ALPES, Saint-Martin-D’Heres (FR)

(72) Inventors: Serge COSNIER, Crolles (FR); Yannig NEDELLEC, Grenoble (FR); Anastasiia BEREZOVSKA, Grenoble (FR); Paulo Henrique MACIEL BUZZETTI, Grenoble (FR)

(21) Appl. No.: 18/700,814

(22) PCT Filed: Oct. 13, 2022
(86) PCT No.: PCT/EP2022/078570

§ 371 Date: Apr. 12, 2024

(30) Foreign Application Priority Data
Oct. 14, 2021 (FR) 2110940

(51) Int. Cl. H01M 4/90; C12N 11/14; H01M 4/86; H01M 8/16

(52) U.S. Cl. CPC H01M 4/9083; C12N 11/14; H01M 4/8605; H01M 4/8663; H01M 8/16

(57) ABSTRACT

An electrode comprising an electronically conductive material at least in part forming a reservoir, the material being permeable and porous; an enzyme compound disposed in the reservoir; and optionally a current collector.

### FIELD OF THE INVENTION

The invention particularly relates to an enzyme electrode, or bioelectrode, and to its uses for the production of electricity, to biocells comprising it as well as to electrical or electronic devices incorporating it. The invention also relates to methods for manufacturing this bioelectrode as well as to assemblies comprising at least two bioelectrodes according to the invention. An electrode according to the invention can also be used to carry out non-enzymatic reactions such as the production of hydrogen by reduction of protons in aqueous medium or the electrochemical reduction of CO₂.

### PRIOR ART

Fuel cell technology is based on the conversion of chemical energy into electronic energy. An organic molecule such as glucose is one of the most important sources of energy for many living organisms and can be considered as a safe, easy to handle, biodegradable biofuel because consumable. Enzymatic biofuel cells (also called biocells) use enzymes to produce energy or electrical power from biological substrates such as methanol, glucose or starch.

Biofuel cells convert biofuel in the presence of enzyme compounds, which produces power. The most well-known biocells function by glucose oxidation (GBFC) are such cells that convert glucose by oxidation at the anode to produce power using an enzyme incorporated therein and having a catalytic function of the reaction. The function of the cathode is generally to reduce oxygen and may or may not comprise an enzyme catalyzing this reaction.

Enzymes are promising alternatives to noble metal-based catalysts since most of them are operational at neutral pH and at room temperature and offer little or no toxicity, which is not the case of other metal-based catalysts. Therefore, biological fuel cells offer an interesting means for providing an environmentally friendly and sustainable energy to electronic devices, in particular to small portable, and/or single-use devices, for applications such as healthcare, environmental monitoring, biodefense, etc.

Given that enzyme-based fuel cells, or biocells, can function using substrates (such as glucose) that are abundant in biological fluids (saliva, blood, urine), of animal or plant origin (fruit juice), etc., as activator and/or fuel. In this context, the terms “fuel” and “biofuel” are interchangeable. In addition, these cells may also make use of environmental effluents (for example glucose and oxygen) while having power densities that are often greater than microbial power densities.

One of the important features of biocells is a small size (for example from 1 to 10 cm² of surface area), or even a very small size (less than 0.5 cm² of surface area) in order to be able to replace the “button” type cells often used in disposable devices. In addition, they must advantageously be of low weights, and preferably, inexpensive. Fuel cells, therefore, offer an interesting proposal for increasing the power or self-power of implantable or portable miniaturized devices [1, 2, 3].

Biocells are confronted with two major technological barriers that currently block their development, namely their short life, and to a lesser extent, their low output power. The low stability of enzymatic cells is linked to the deactivation of immobilized enzymes and seems ineluctable. Indeed, the energy-generating elements (bioanode and biocathode) of these biocells are based on the immobilization of various redox enzymes on the surfaces of electrodes for their electrical connection. This attachment of enzymes can be obtained by physical capture or by chemical grafting or affinity interactions. The first leads to a denaturation process due to the capture process itself and to the non-biocompatible environment. In addition, the activity of the captured enzyme may be affected by the permeability and the hydrophobia of the host structure, and the steric strains that block its conformational flexibility. As regards the process of chemical grafting and of affinity bonding, better access of the substrate to the immobilized enzyme may be obtained but the amount of biocatalyst is limited to an almost single layer at the modified electrode-solution interface thus significantly limiting the power. In addition, the electrical connection of enzymes by redox mediators is difficult due to the access to the active site of the enzyme that may be blocked by the immobilization of the latter.

A strategy developed recently consists in using a dialysis bag to contain the elements of the bioelectrode (enzymes/conductor) in a liquid medium, this making it possible to avoid immobilizing the enzyme covalently and to preserve its activity. Thus, Cinquin et al.¹ proposes the use of electrodes where the enzymes and the mediators are compacted in graphite disks but that are not covalently fixed therein. These electrodes are disposed in dialysis bags. However, in such devices the connection of the enzyme by the redox mediator is random. The enzyme and the mediator are immobilized by compression and can hardly move to connect whereas they must be very close to the active site. In addition, only the enzymes located close to the surface of the compressed graphite disk will be active, as the other enzymes are not accessible to the substrates. WO2019234573 also describes pellet-type electrodes that offer the same limitations. These limitations are also present in the biocells described in FR3103325 where the enzymes are disposed on carbon nanotube sheets by pipette deposition. None of these devices have reservoirs internal to the electrode, simply an association of reagents.

Hammond et al.² also proposed bioanodes comprising conductive substrate disks disposed in an aqueous suspension comprising enzymes and nanoparticles of non-immobilized specialist mediators. The suspension can diffuse through a wall made of a dialysis membrane, this making it possible to prevent the escape of mobile active compounds (enzymes, coenzymes, mediators) and the intake of glucose.

Li et al.³ describe a bioanode comprising an enzyme system/mediator/conductor in the form of an aqueous suspension or “slurry”. The anode consists of:
an enzyme system consisting of glucose oxidase (GOx) and its Flavin Adenine Dinucleotide (FAD) cofactor;
Ti₃C₂ MXene nanosheets (a graphene-type lamellar conductor);
carbon felt; and
an electron mediator based on terephthaldehyde (TPA) reticulated with aminoferrocene (amino-FC).

The aqueous suspension is conserved in a dialysis bag of dimension 20×14×4 mm, having a molecule retention threshold of 100 Da. Multi-walled carbon nanotube MWCNT particles do not provide acceptable results due to their poor dispersions in the liquid. In addition, some mediators are inadequate because their sizes do not allow them to be effectively retained by the dialysis envelope.

However, due to their structures, these devices have many drawbacks. First of all the size, in particular the volume, of such devices is increased, which is the opposite of one of the advantages particularly sought. The fragility and the porosity of the dialysis film and/or the presence of liquid do not allow easy storage and/or handling. The components of the electrode must be suitable for use in the form of liquid dispersion and are therefore highly specialized and therefore expensive. Finally, the manufacture of these electrodes is rendered particularly delicate by the presence of liquid and porous and flexible films.

Thus, generally the aim of the invention is particularly to solve the problem of providing an electrode for a biofuel cell, in particularly of design allowing it to be used in devices of small dimensions, which is inexpensive (e.g., of the button or “coin” cell type) and/or easy to store and/or use, while having an optimized power.

Another aim of the invention is to increase/maximize the power of a biocell while minimizing its size and the total enzyme mass used.

The invention particularly aims to combine the presence of enzymes or of catalysts having an improved activity, because being able to be placed in solution, in a device that is easy to operate, manufacture and/or store while respecting the environment. Indeed, the use of toxic and/or expensive compounds can be minimized, or even eliminated, during the production of an electrode according to the invention.

### DESCRIPTION OF THE INVENTION

Ingeniously, the invention proposes an electrode having a reservoir, of which the reservoir comprises a conductive material. Thus, one object of the invention is an electrode comprising:
an electronically (or ionically) conductive material at least in part forming a reservoir, said material being permeable and porous, and/or comprising carbon nanotubes;
an electrochemical reaction catalyst, and in particular an enzyme compound, disposed in the reservoir; and
optionally, a current collector.

The term “electrode” is used in a broad sense and not only designates the electron (or ion) conductor that can capture or release electrons but also by extension the anode or cathode compartment of a half-cell.

The term “permeable” is used to indicate that the voids that the conductive material contains are continuous and allow the diffusion of liquid and in particular of water.

The term “porous” is used to describe a material comprising voids (pores), of which the size allows the passage of liquid. The material according to the invention must be of a porosity allowing the passage of the substrate of the electrochemical reaction in question (for example oxygen, glucose, etc.) while allowing the retention of the catalytic entities used such as an enzyme, a coenzyme, an orientor, and/or a redox mediator, etc. In the bioelectrodes that are a preferred aspect of the invention, the average size of the pores, that is to say its porosity, is measured by pressurized nitrogen adsorption/resorption. This porosity is preferably chosen in a range of 0.1 nm to 5 nm, preferably of 0.2 nm to 3 nm and advantageously of 0.3 to 2 nm. The standard measurement technique used is the nitrogen adsorption-desorption manometry implementing the BJH (Barrett-Joyner Halena) method derived from the BET (Brunauer Hemmet Teller) specific surface measurement. The sample is vacuum degassed in advance in order to eliminate any trace of residual moisture or any solvent that could distort the measurement at temperatures between room temperature and 80° C. max. for a minimum of 1 h and a maximum of 4 h. The apparatuses used may be of the Micromeritics brand (ASAP 2020) from Micromeritics Instrument Corporation (CA) or Quantachrome (NOVAtouch), AT.

The term “reservoir” designates a physical space, such as a cavity, making it possible to place a compound, and in particular an electrochemical reaction catalyst such as an enzyme compound, in reserve. Since it is placed in reserve the compound does not immediately react when the conditions of the electrochemical reaction are present at the electrode.

The conductive material according to the invention may comprise, or consist of, a solid, preferably recyclable, agglomerate such as carbon felt, microporous carbon, carbon nanotubes, active carbon, mesoporous carbon, carbon black, conductive polymers and mixtures thereof. The carbon nanotubes are particularly adapted to the manufacture of a conductive material for the electrode according to the invention. This material may be an agglomerate based on single-walled carbon nanotubes (CNT) or more advantageously multi-walled carbon nanotubes (MWCNT), because they offer excellent porosity associated with excellent conductivity. According to a particular aspect of the invention, the electrode according to the invention is not contained, in part or in whole, in a dialysis membrane.

“Carbon nanotube” means a carbon nanotube of which at least one dimension is less than 1,500 nm. Preferably, the carbon nanotubes have a length (L) to diameter ratio denoted L/diameter between 100 and 5,000. Preferably the carbon nanotubes have a length of approximately 1.5 μm and/or for example a diameter less than approximately 20 nm. The average diameter of the nanotubes that is particularly preferred for implementing the invention and that gives the best results is less than 10 nm and in particular may range from 1 to 8 nm. Carbon nanotubes having diameters in the order of the micron, or even in the order of 75 to 200 nm are not the most performant.

The conductive material may comprise such materials or essentially consist thereof. “Essentially consist of” means that the material consists of more than 90%, preferably more than 95%, by mass of this conductive material. Such a material associates a very good porosity and a significant ease of manufacture at very low cost and makes it possible to optimize the interactions between the catalyst and the substrate, or fuel of the electrochemical reaction. Thus, according to one aspect of the invention, the conductive material may not comprise other materials and in particular it may not comprise polymeric materials (binder) that may affect the performances of the electrode. However, the conductive material may comprise one or more compounds that take part in the electrochemical reaction taking place at the electrode. For example, an orientor or a redox mediator (cf. infra) may be present in the material. Preferably this material is adsorbed on a conductive material so as to preserve a certain mobility.

The conductive material is preferably in the form of a sheet, a film or of thin leaves. The sheet, the film or the leaf may have a thickness less than 1 mm, preferably from 50 to 500 μm, in particular from 100 to 400 μm, for example approximately 250 μm. The presentation in the form of thin sheets allows easy handling, and in particularly cutting, and therefore manufacturing of the electrode. Thus, the electrode can easily have varied forms. A cylindrical or circular shape is preferred but many other shapes may thus be envisaged.

The conductive material forms at least one part of the reservoir of the electrode. It may form it completely or mostly but according to a particular alternative embodiment of the invention it only forms part thereof.

Preferably the conductive material is hydrophilic, that is to say that in the presence of an aqueous liquid, the contact angle at the equilibrium of a drop of this liquid on the surface of the material is less than 90°. The contact angle may be measured by the sessile drop method using a camera-assisted goniometer. The measurement is taken at atmospheric pressure and at room temperature. The sample undergoes no prior treatment. The equipment used may be for example a DATA Physics OCA 35 Microdrop from DataPhysics Instruments GmbH (Germany). The measurement is taken using a goniometer consisting of a CCD (Charge Coupled Device) camera, a highly magnifying optical system and a light source. The liquid deposition system is automated to obtain better reproducibility in the measurements. The volume of the drop is generally from 1 to 5 μL. For the characterization of the wetting of textured surfaces, the contact angle is measured on the left and right sides of the drop in order to calculate an average of the contact angle and the standard deviation of the series of measurements. For the image analysis, we use the ellipse or circle equation method where the entire contour of the drop is correlated to a circle or an ellipse by applying Young's modulus. Preferably the contact angle is less than 45°, preferably less than 30°, advantageously less than 10° C., also more advantageously less than 5°.

According to a preferred embodiment due to its significant simplicity and adaptability, the reservoir comprises, or consists of, two sheets made of conductive material joined together using an adhesive. The term “adhesive” is used to designate any material making it possible to join together by contact conductive material walls. This term covers products that may be designated by other names, for example “ink”, but of which the liquid, gelatinous or pasty consistency allows deposition on parts then bonding them subsequently together by contact then drying, hardening and/or polymerization. Of course, it is preferred that this adhesive is not insoluble in liquids such as water or aqueous liquids. This adhesive may be chosen in the group consisting of vinyl adhesives (white liquid glues), acrylics, aliphatics, cyanoacrylates, polyurethanes, epoxies, neoprenes, hot melt adhesives, thermoplastic resins, silicones (for example polydimethylsiloxanes (cPDMS)) and mixtures thereof. Preferably, this adhesive is itself conductive and/or contains conductive particles such as carbon or graphite particles. A thermoplastic resin containing fine graphite or polydimethylsiloxane particles is particularly adapted for implementing the invention.

It should be noted that for implementing the invention the reservoir does not need to be formed mostly of a permeable and porous conductive material, and/or comprising carbon nanotubes. Indeed, a reservoir of which less than 50% of the internal surface consists of said materials allows good results to be obtained. The rest of the walls of the reservoir may comprise one or more conductive materials that have features other than porosity and permeability, such as for example flexibility and/or solidity. Such materials may be sheets of nanotubes with a polymeric binder or of vitreous carbon, of carbon fibers, etc. However, it is preferred that more than 30% of the internal surface of the reservoir and in particular between 30% and 80%, in particular between 40 and 60% of the internal surface of the reservoir consists of a permeable and porous conductive material, and/or comprising carbon nanotubes. According to a preferred alternative embodiment of the invention, the internal surface of the reservoir consists of more than 50%, preferably more than 75%, and more particularly more than 85% of the permeable conductive material.

Thus, it is possible to obtain an electrode having a reservoir according to the invention of a very small dimension (volume) and in a particularly easy way particularly by gluing two conductive sheets together so as to form a reservoir of which a part, in particular a part of the height, is made of the adhesive material. Such an electrode having a microreservoir is a particularly advantageous alternative embodiment of the invention. Preferred dimensions of this electrode particularly comprise:
a total internal volume of the reservoir ranging from 10 μL to 500 μL, preferably from 10 μL to 100 μL and advantageously from 10 μL to 50 μL;
a height of the reservoir of 25 μm to 200 μm, preferably of 40 μm to 100 μm, for example of 60 μm+10 μm;
a thickness of the electrode ranging from 400 μm to 5 mm, preferably 500 μm to 2 mm, for example around 1 mm; and/or
an outer surface area of the electrode that may vary from 0.5 to 10.0 cm², preferably from 0.8 to 7 cm², for example approximately 1 cm².

Selecting a reservoir of very small volume (for example a few tens of microL) makes it possible to have a strong concentration of catalytic compound in solution (for example 0.2 g/mL) for a very low amount of catalytic compound (a few mg) and therefore a very low production cost.

In particular, the electrodes according to the invention can take the form of small (1 to 2 cm in diameter), or even very small (at least 0.5 cm in diameter), pellets, for example circular or polygonal. Such electrodes can have a thickness varying from 5 mm to 0.1 mm, for example 0.25 mm.

The reservoir of the electrode preferably contains a catalyst, the term “catalyst” is used in its acceptance of catalytic entity to designate one or more compounds making it possible, alone or combined, to catalyze, accelerate and/or promote the electrochemical reaction taking place at the electrode. In the context of biocells, it may concern an enzyme compound, such as an enzyme and/or an enzyme cofactor, a compound absorbing the harmful species, an orientor (a compound making it possible to orient the enzyme when it approaches the conductive material to facilitate the electron transfer) and/or a redox mediator. When proteins or protein derivatives having an enzyme function are used, these enzyme compounds comprise native proteins as well as the derivatives, mutants and/or functional equivalents thereof. This term in particular extends to proteins that do not substantially differ at the structure and/or the enzyme activity. The enzyme compound may be a combination, or association, of enzymes, these able to catalyze the same reaction or different reactions.

When the electrode is a bioanode and/or the substrate of the reaction is glucose, the enzyme may be a glucose oxidase (GOX), preferably with its Flavin Adenine Dinucleotide (FAD) cofactor or a dehydrogenase such as flavin adenine dinucleotide-Glucose DeHydrogenase (FAD-GDH) (EC 1.1.5.9). If the use of GOx implies the production of oxygenated water (harmful species) a compound contained in the reservoir of the anode may be a catalase.

When the electrode is a bioanode and/or the substrate is oxygen O₂, the enzyme may be an oxygen-reducing enzyme, and more particularly bilirubin oxidase (BOD) (CAS number 80619 January 8; April 2018), a polyphenol oxidase (PPG), or a laccase (LAC), which may advantageously be associated with an orientor, a protoporphyrin IX, such as hemin.

When the substrate of the reaction is oxygen O₂ and glucose, the reservoir of the electrode may contain an oxygen-reducing enzyme, and more particularly a glucose oxidase (e.g., Aspergillus niger), in association with an enzyme reducing hydrogen peroxide in water, such as a peroxidase (for example, peroxidase from horseradish). This enzyme combination may be advantageously associated with a redox mediator such as ABTS.

The terms “biocathode” and “bioanode” refer to the presence of biological material, for example an enzyme, in the structure thereof or close thereto.

When a redox mediator is used, it may also comprise a molecule, particularly aromatic, acting as a redox mediator or orientor, such as 1,4-naphtoquinone, to improve the electron exchanges. Molecules chosen in the group formed by 9,10-phenanthrenequinone, 1,10-phenanthroline-5,6-dione, 9,10-anthraquinone, phenanthrene, 1,10-phenanthroline, 5-methyl-1,10-phenanthroline, pyrene, 1-aminopyrene, 1-pryenebutyric acid, ABTS, protoporphyrine IX such as hemin, and mixtures of two or more thereof may also be considered. The use of such compounds is particularly advantageous in the case of enzyme systems comprising an FAD-GDH or a GOx.

The catalyst, in particular when this is an enzyme, is advantageously disposed in the reservoir in solid form, in particular powder. This form not only allows for simple and easy manufacture but also makes it possible to obtain an electrode that can be stored prior to its use and that can easily provide a high concentration of catalyst.

According to a particularly preferred aspect, the concentration of catalyst, in particular of an enzyme, when diluted in liquid medium such as water, is high. In particular, this concentration may be 0.01 g/mL to 1 g/mL, preferably 0.05 g/ml to 0.5 g/mL, for example be approximately 0.2±0.1. Alternatively or additionally, this concentration may be 0.5 mM to 5 mM, preferably 1 mM to 3 mM, for example approximately 2.5 mM±0.1.

When the conductive material at least in part forming the reservoir, and being permeable and porous, and/or comprising carbon nanotubes is a solid agglomerate, it may advantageously be combined (“functionalized”) with an enzyme compound or with one forming part of an enzyme system. In particular a compound such as an orientor or a redox mediator (cf. supra) may be mixed with the conductive material. The mixture can be carried out during the manufacture of the material or adsorbed thereon, for example by drop casting.

However, according to a particularly advantageous aspect of the invention, the conductive material of the electrode according to the invention is not functionalized by an orientor or a redox mediator. For example, it is not functionalized by ABTS and/or by one of the abovementioned compounds. If an orientor or a redox mediator is present, it may be disposed directly in the reservoir, for example in solid form, such as a powder, without having to be associated, or bonded, with the conductive material. This ease of use is very advantageous since it makes it possible to eliminate the need for a manufacturing step.

The electrode according to the invention may also comprise a current collector. This may be in the form of layers, tabs, films and/or threads. Advantageously, it has a low thickness, a high thermal and/or electrical conductivity and may comprise, or (substantially) consist of, highly oriented and preferably flexible graphite. Thus, it is also possible to use a sheet, or a tab, made of a pyrolytic graphite sheet. The use of graphite is advantageous due to the fact that it combines stability, lightness and thermal and electrical conductivity. Its thickness can be chosen as ranging from 10 to 500 μm, preferably from 17 to 300 μm, and advantageously from 40 to 2,000 μm. Its thermal conductivity (in the longitudinal plane of the electrode) may be 100 to 1,000 W/(m·K), preferably 100 to 1,950 W/(m·K) and advantageously 100 to 1,350 W/(m·K). This layer may also have an electrical conductivity greater than 5,000 S/cm, preferably greater than or equal to 8,000 S/cm, for example around 10,000 S/cm. However, it may have a higher conductivity, for example approximately 20,000 S/cm, in particular if the thickness of the layer is less than 40 μm. This layer may also have a heat resistance, for example a resistance to a temperature of more than 200° C., advantageously of more than 300° C., for example of approximately 400° C.

Another object of the invention is a cell, and in particular a biocell, particularly of the fuel cell type, comprising an electrode according to the invention. The biocell may comprise an electrochemical cell, said electrochemical cell comprising an anode and a cathode. The anode or the cathode, and advantageously the two, are advantageously an electrode according to the invention. Said biocell may further comprise means for electrically switching on said biocell with an electrical receiver, said electric switching means allowing current to flow between the anode and the cathode.

The term “cell” is used in its broadest sense. Thus “cell” is understood, among other things, to be a device only having a single electrochemical cell and/or a chargeable or non-chargeable device. A cell comprising a stack of a plurality of electrical chemical cells is envisaged to obtain the required voltage.

Advantageously, the cell according to the invention may be of varied shape and/or of small size. In particular, it may only occupy a volume less than or equal to 2 cm³, preferably less than or equal to 1 cm³, or even less than or equal to 0.75 cm³. It may particularly be designed to be able to replace “button type” batteries. The distance between the electrodes is easily adapted by the person skilled in the art, but it is noted that this distance may vary from 1 to 10 mm without this variation having consequences on the performances of the cell.

The cell according to the invention may comprise switching means such as terminals (for example at least one positive terminal and at least one negative terminal) which may connect the current collectors with the outside of the biocell. Such terminals make it possible to allow the input and output of the electric current. These terminals may be a portion of the switching means that are suitably sized and positioned.

The cell according to the invention may comprise a separating and porous membrane, electrically insulating, and permeable to the liquid medium, which is disposed between the anode on the one hand and the cathode on the other hand. This membrane allows the passage particularly of ionic species and, advantageously, substrates between the anode and the cathode.

For some uses, the cell according to the invention may advantageously comprise an outer coating that may be a protective support, layer, or film, which partly covers the electrochemical cell(s) of the device. This is preferably flexible, adhesive, non-toxic, chemically stable, electrically insulating, insensitive to radiation and/or has a wide operating temperature range (for example from −150° C. to 200° C., or even approximately 260° C.). This coating, or outer protective film, may comprise, or (substantially) consist of a glass fiber fabric impregnated with a relatively inert material such as perfluorinated polymeric material of the PTFE (polytetrafluoroethylene) type or a silicone-based material. The PTFE can be Teflon® from Du Pont de Nemours, Fluon® from Asahi Glass, Hostaflon® from Dyneon. The film or coating is preferably impregnated with more than 50% by weight of said material, advantageously 50 to 70%, preferably 57 to 64% relative to the total weight of the film. Its thickness may be a few tenths, or even hundredths of millimeters. For example, it may be chosen in a range of 0.03 to 0.50 mm, preferably of 0.05 to 0.30 mm and preferably of 0.06 to 0.14 mm, for example be 0.07 mm. According to a preferred aspect of the invention, the coating, or protective film, comprises an adhesive layer, preferably water resistant, allowing it to adhere to the outer surface of the electrochemical cell(s) of the biocell according to the invention. Another material that can be used as outer coating may be of the non-woven adhesive tape type comprising a layer of synthetic fibers (for example a polyester/rayon mixture) and an adhesive layer (for example acrylate-based). This type of material generally for medical use is perfectly suitable as an outer coating.

According to a particular aspect, this protective film may be affixed directly on a face of an electrode or of the cell. According to another preferred aspect, this outer coating, which is preferably flexible and insulating, comprises one or more openings positioned and dimensioned so as to allow the access of a liquid at the anode and/or the cathode. This opening can be precut in the coating. Additionally, or alternatively, this opening may be formed due to the fact that the coating does not completely surround the biocell comprising the electrochemical cell(s) but leaves an opening giving access to these elements.

Thus, the cell according to the invention may advantageously comprise an outer coating, preferably flexible, insulating and/or impermeable to liquid comprising openings positioned and dimensioned so as to allow the access of a fluid and in particular of a liquid, for example an aqueous liquid.

According to one aspect of the invention, the electrochemical cell may comprise a series of layers, preferably thin, flexible and/or mechanically robust, preferably forming a self-supporting multilayer (or multi-lamellar) stack. The shape and/or the dimension of these layers, and particularly the presence of at least one opening and/or recess, are advantageously determined so as to form, or allow, an electrical connection, an inlet for the substrates. These layers comprise the anode, the cathodes, any separating layers and the switching means, as described in the present application.

Another object of the invention is a method for manufacturing an electrode as described in the present application. This method comprises the positioning and the joining of the constituent elements of said electrode. This method may comprise the use of at least one material (in particular in sheet form) and of an adhesive as described above and comprises the step of positioning on this material, a wall, continuous or not, of adhesive to form a cavity then of sealing this cavity by means of a permeable and porous conductive material, and/or comprising carbon nanotubes, in order to form, at least partly, a reservoir. This method may also comprise at least one of the following steps of:
filling the reservoir by a catalyst such as an enzyme; and
positioning a current collector.

Preferably the positioning is a superposition of said elements.

The invention also relates to a biocell as described in the present application and further comprising an aqueous liquid, said liquid optionally comprising a biofuel. The fuel may however be already present in the device in a dry and/or solid and/or non-solubilized form and/or may migrate to the enzyme sites as described in patent publications FR1855014 and WO2019234573.

When the aqueous liquid is added, it diffuses inside the reservoir and the catalyst (particularly the enzyme) present in the reservoir is dissolved in the liquid which allows electrochemical exchanges to take place. Alternatively or additionally, the added liquid comprises the biofuel. This can be, for example, a physiological liquid such as blood, urine or saliva or an alcoholic or glucose drink.

Another object of the invention is a method for activating the electrode comprising placing an electrode as described in the present application in the presence of a liquid, an aqueous liquid, optionally comprising a fuel such as sugar (for example glucose, fructose, saccharose and/or lactose, etc.), starch and/or ethanol.

Another object of the invention is an apparatus comprising a biocell according to the invention, and an electrical receiver (that is to say an apparatus that uses (receives) electric current), said biocell being electrically connected to said electrical receiver. Such an apparatus can be a test, in particular a test of the biological fluid: for example, a pregnancy test or a blood sugar level test. It may also be an apparatus for emitting an alert signal, for example when the biocell is in association with a diode. In view of its very low cost, an alert device for changing protective layers (for example for urinary leaks) is envisaged. The apparatus may also be in the form of a patch for the skin, the supply of the biocell taking place through perspiration that contains lactate and oxygen. In particular, due to the presence of glucose and oxygen in extracellular fluids (blood and interstitial fluid), enzyme cells according to the invention can be used in implantable devices including devices for feeding into the human body, implanted medical devices such as stimulation electrodes, cardiac stimulators, pumps, sensors and bionic implants, etc. A cell according to the invention can also be used in a GPS locating apparatus that may be used for mapping the movement of endangered animal species (tigers, elephants, etc.).

Alternatively or additionally the biocell (and/or the device) according to the invention can be incorporated into an electronic device with electronic display and/or light emission.

More generally the apparatus according to the invention is of the type operating with button type batteries using metallic derivatives, such as an apparatus for point-of-care testing (POCT), the Internet of Things (IoT) or an environmental sensor.

Such an apparatus according to the invention can advantageously be disposable, biodegradable and/or for single use.

Another object of the invention is a kit for the manufacture of a biocell as described in the present application and that comprises a biocell as described in the present application, associated with instructions for use.

Another object of the invention is the use of a biocell according to the invention for the generation of an electric current.

Another object of the invention is an electrochemical cell as described above.

Another object of the invention is the use of an electrode according to the invention in the manufacture of cells, biocells, devices and apparatuses as described in the present application.

According to another aspect of the invention, the electrode having an internal reservoir can be used for chemical reactions other than reactions using biochemical compounds such as enzymes. An electrode according to the invention can be used to carry out non-enzymatic reactions such as the production of hydrogen by reduction of protons in aqueous medium or the electrochemical reduction of CO₂. The catalysts or mediators of the reaction can be organic or metallorganic compounds soluble or partially soluble in an aqueous medium that will be captured such as enzymes in the (micro) cavity. For example, 5,10,15,20-tetrakis(4-sulfonatophenyl)-iron porphyrin could be used for the electrochemical reduction of CO₂ and a rhodium complex: [RhIII(tpy)(CH3CN)Cl2](CF3SO3) for the electrogeneration of H₂.

### BRIEF DESCRIPTION OF THE FIGURES

The invention will be better understood upon reading the following description given purely by way of example and referring to the appended drawings, wherein:

FIG. 1 is an exploded perspective view of an example of configuration of a bioelectrode according to the invention.

FIG. 2 is a transparent and perspective schematic view of the bioelectrode of FIG. 1.

FIG. 3 is a photographic top view of an electrode as shown in FIGS. 1 and 2 during manufacture.

FIG. 4 is a photographic top view of a step of manufacturing an electrode as shown in FIGS. 1 and 2, subsequent to the step of FIG. 3.

FIG. 5 is a photographic top view of a step of manufacturing an electrode as shown in FIGS. 1 and 2, subsequent to the step of FIG. 4.

FIG. 6 is a laser microscopic view of a cross-section of an electrode according to Example 2.

FIG. 7 is a cyclic voltammetry diagram of the bioanode of Example 1, with and without the presence of glucose

FIG. 8 is a cyclic voltammetry diagram of the biocathode of Example 2, with and without the presence of oxygen.

FIG. 9 shows the evolution of the catalytic current at 0.3 V as a function of time by maintaining an O₂ bubbling of the electrode of Example 3.

FIG. 10 shows a diagram of the biocell of Example 4.

FIG. 11 shows the biasing/power curve of the biocell having a cavity of Example 4 and the power obtained during discharging 10 s in a phosphate buffer pH 6.5 in the presence of 100 mM of O₂-saturated glucose.

FIG. 12 shows the evolution of the maximum power (obtained by linear scanning voltammetry at 0.2 mv/s) as a function of time in phosphate buffer (pH 6.5) medium containing 100 mM of glucose and saturated in O₂ of the biocell of Example 4.

FIG. 13A shows the H₂O₂ detection curve by HRP-catalytic reduction current vs [H₂O₂]@−0.2V vs Ag/AgCl (saturated argon) of Example 5.

FIG. 13B is an enlargement of a portion of the curve of FIG. 13A.

FIG. 14 is a representation of the operation of a GOx-HRP-ABTS bi-enzyme cavity electrode according to Example 6 of the invention.

FIG. 15A makes it possible to observe the indirect detection of H₂O₂ produced by GOx by successive injections of glucose-catalytic reduction of H₂O₂ by HRP @0V vs Ag/AgCl; PB 7.4—ambient air)—FIG. 15B shows the calibration curve established from FIG. 3 (A)—catalytic current for reduction of H₂O₂ by HRP as a function of the glucose concentration in solution.

### EXAMPLES OF EMBODIMENTS

### Protocol

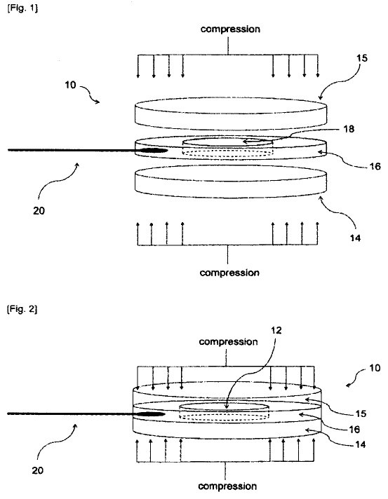

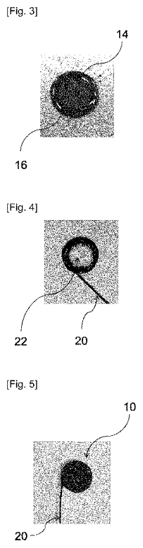

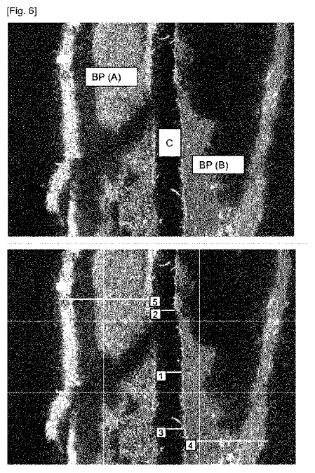

A base configuration of electrodes according to the invention is exemplified in FIG. 1 (exploded view) and FIG. 2. According to this example, the electrode 10 comprises a reservoir 12. Here, the reservoir 12 consists of a disk 14 of Buckypaper around the periphery of which is disposed a circular line of adhesive 16 defining an inner cavity 18. This inner cavity 18 and the line of adhesive 16 are covered by another disk of Buckypaper 15, covering the cavity 18 and this line of adhesive 16 and thus defining the reservoir 12. Thus, FIG. 3 shows a top view of a disk 14 of Buckypaper of 12 mm in diameter around the periphery of which is disposed a circular line of adhesive 16 defining an inner circular cavity 18 of 7 mm in diameter. An electrical wire 20 is disposed in/on the adhesive and a powder enzyme 22 is disposed in the cavity 18 (FIG. 4). This enzyme powder 22 and the cavity 18 containing it are then covered with a sheet of Buckypaper 15, which, adhering to the adhesive 16, seals the inner cavity 18 and transforms it into a reservoir 12.

### List of Materials Used in the Samples.

Multi-walled carbon nanotubes (CNTS) from Nanocyl NC7000™; Nanocyl SA, Rue de l'essor, B-5060 Sambreville, Belgium.
Commercial buckypapers: NTL Composites of reference NTL-12217, 60 g·sm MWCNT Blend, nanotechlabs, 409 W. Maples ST, Yadkinville, NC 27055,
Carbon adhesive: LOCTITE EDAG 423SS E&C; Henkel France S.A.S, 161 Rue de Silly, 92100 Boulogne-Billancourt, France.
Enzymes:
Bilirubin oxidase (BOD) from Myrothecium verrucaria; Amano 3 d′Amano Enzyme Inc., U.S.A.
Glucose dehydrogenase dependent FAD from Aspergillus sp; Sekisui Diagnostics, Itd, UK.
Mediators, orientor:
PQ: Phenanthrene quinone; CAS 84-11-7 from Fluka AG
PLQ: 1,10-Phenatroline-5,6 dione; CAS 27318-90-7 from Sigma Aldrich
1,4 NQ: 1,4 Naphthoquinone; CAS 130-15-4 from Sigma Aldrich Hemin: BioXtra porcine, CAS 16009-13-5 from Sigma Aldrich.
   Preparation and Shaping Buckypaper from Carbon Nanotubes

The carbon nanotubes (CNTs) (NC7000) are dispersed in DMF (ratio 1/1 mass (mg)/volume (mL) and subjected to an ultrasonic bath for 1 h 30. The ultrasonic bath used is a Fisher Scientific FB15050. The ultrasonic frequency is 37 kHz for an effective power of 80 W RMS. The dispersion is then filtered on vacuum Buchner, (PTFE filter 0.45 μm) until evaporation of the solvent for a minimum period of 3 h. The solid film is then rinsed with water (H2O), vacuum dried, then dried in ambient air for one night under compression. The buckypaper (A) can be sized to the required dimension using a punch, for example in the shape of a disk of 12 mm in diameter. Its thickness is approximately 200-250 μm.

### Example 1 Anode: Catalytic Oxidation of the Glucose

An electrode was manufactured according to the protocol described above. The buckypapers (A) and (B) were functionalized by drop-casting 200 μL of PLQ/CH₂Cl₂ (5 mM). Commercial buckypaper (B) is a composite buckypaper comprising a binder making it possible to give a certain flexibility to the electrode.

The spacer material is LOCTITE EDAG 423SS E&C carbon adhesive, and the powder enzyme deposited in the cavity is FAD-GDH 4 mg. The anode is disposed in a beaker containing a phosphate buffer solution at pH 7 then in a solution containing glucose (100 mmole·L⁻¹) solution in the presence of the same phosphate buffer.

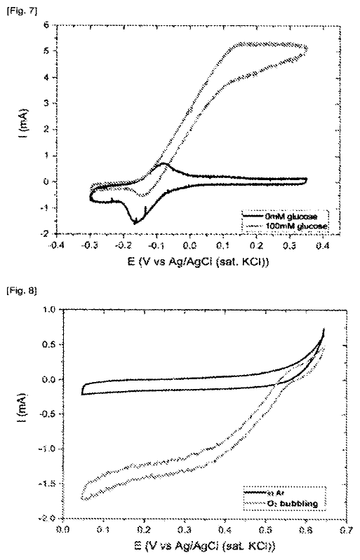

Coupled with an Ag/AgCl reference electrode (Saturation of KCl) and with a Pt counter-electrode (scanning speed 1 mV·s⁻¹), a cyclic voltammetry recording (FIG. 7) shows that in the presence of glucose, an anodic catalytic current due to the oxidation of the glucose via the electrical connection of the enzyme appears. The electrical connection of the enzyme is ensured by the redox mediator (PLQ) adsorbed on the surface of the carbon nanotubes, which allows the indirect electron transfer with the enzyme.

### Example 2 Cathode: Catalytic Reduction of Oxygen

An electrode was manufactured according to the protocol generally described above. However, only the buckypaper (BP (A)) was functionalized by hemin (0.6 mM). In addition, the functionalization was carried out during the manufacture of the buckypaper A. The nanotubes were dispersed in DMF in a ratio of 1/1 by mass (mg/mL). In this solution, hemin was added in an amount necessary for obtaining a concentration of 0.6 mM (or 0.6 mmole·L⁻¹). This nanotube/hemin/DMF solution is then filtered on Buchner according to the method described above.

FIG. 6 is a laser microscopic view of a cross-section of this electrode. The cavity C, of heights \textcircled{1}, \textcircled{2}, and \textcircled{3}, is visible between the thickness 5 of the buckypaper A (B(A)) and the thickness 4 of the buckypaper B (BP(B)). The BOD enzyme present in the cavity does not appear due to the contrast used. The dimensions \textcircled{1}, \textcircled{2}, \textcircled{3}, \textcircled{4}, and \textcircled{5} of the buckypaper A (B(A)) and of this electrode are given in Table 1 with reference to FIG. 6.

TABLE 1

| | Seg. | Distance |
|---|---|---|
| \textcircled{1} | 2-pt dist | 62.659 μm |
| \textcircled{2} | 2-pt dist | 55.085 μm |
| \textcircled{3} | 2-pt dist | 67.479 μm |
| \textcircled{4} | 2-pt dist | 200.400 μm |
| \textcircled{5} | 2-pt dist | 226.536 μm |

The spacer material is LOCTITE EDAG 423SS E&C carbon adhesive, and the powder enzyme deposited in the cavity is BOD in an amount of 2 mg. The anode is disposed in a beaker containing a phosphate buffer solution at pH 7.4 then in a solution with oxygen bubbling in the presence of the same phosphate buffer.

Coupled with an Ag/AgCl reference electrode (Saturation of KCl) and a platinum counter-electrode (scanning speed 1 mV·s⁻¹). A cyclic voltammetry recording (FIG. 8) shows the appearance of a strong cathodic current that reflects the connection of the enzyme by direct electron transfer with the carbon nanotube sheets: BP (A)+hemin/Loctite/BP (B) adhesive-phosphate buffer pH 7.4 O₂ bubbling.

### Example 3: Stability of a Biocathode According to the Invention

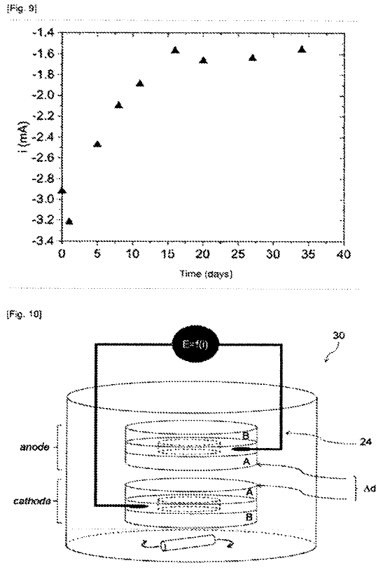

The cathode of Example 2 was reproduced with increased dimensions (external diameter 30 mm, internal diameter 13 mm) and the same amounts of hemin and of BOD as in Example 2 so as to observe its stability via the recording of the catalytic current at 0.3 V in a phosphate buffer pH 6.5 (FIG. 9) by maintaining an O₂ bubbling. The performance of the electrode appears to be, to a certain extent, dependent on the available conductive surface area. With an identical amount of enzyme, it is therefore possible to increase the electro-enzymatic current by increasing the surface area of the electrode.

A reduction of the electro-catalytic activity appears during the first 10-15 days then a stabilization of this current up to 5 weeks. This is fairly remarkable because the biocathodes based on bilirubin oxidase (BOD), in general, have their activity which disappears after a few days or even one week.

### Example 4: Biocell Based on Two Electrodes Having a Reservoir According to the Invention

A biocell (30) according to the invention was produced according to the diagram of FIG. 10 using a bioanode according to Example 1 and a biocathode according to Example 2. Their respective compositions are summarized in Table 2 below:

TABLE 2

| ANODE | CATHODE |
|---|---|
| Buckypaper (A) + 200 μL drop-casting | Buckypaper (A) + hemin 0.6 mM |
| PLQ/DCM (5 mM) | |
| Spacer material (C); LOCTITE EDAG | Spacer material (C); LOCTITE EDAG |
| 423SS E&C Loctite carbon adhesive | 423SS E&C Loctite carbon adhesive |
| Buckypaper (B) + 200 μl drop-casting | Buckypaper (B) without treatment |
| PLQ/DCM (5 mM) | |
| FAD-GDH 4 mg | BOD 2 mg |
| External diameter 12 mm, internal | External diameter 12 mm, internal |
| diameter 7 mm | diameter 7 mm |

These two electrodes are disposed in a beaker containing an O₂ saturated phosphate buffer (24) and containing a glucose concentration of 100 mM, at a pH 6.5-7.4, under magnetic bar agitation. The distance Δd between the two electrodes is 5 mm.

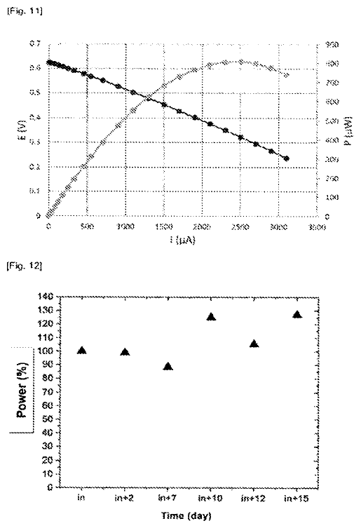

The power obtained during discharging 10 s in a phosphate buffer pH 6.5 in the presence of 100 mM of O₂-saturated glucose is recorded (FIG. 11) and shows a maximum power of the biocell of 807 μW, i.e., 1.048 mW/cm² by taking into account the electroactive surface area for each electrode (0.769 cm²).

The stability of the biocell is illustrated via the evolution of its maximum power (obtained by linear scanning voltammetry at 0.2 mv/s) as a function of time in phosphate buffer (pH 6.5) medium containing 100 mM of glucose and saturated in O₂ (FIG. 12). An increase of the power appears after one week certainly due to the improvement of the permeability of the commercial buckypaper over time; this results in a greater addition of substrates to the enzyme and therefore increases the catalytic current. No reduction is observed after 15 days as opposed to conventional biocells, thus illustrating the advantages of the invention.

### Example 5: HRP/ABTS Bioelectrode

An electrode is produced on the principle of the preceding examples. In order to produce a bi-enzyme electrode, two sheets of buckypaper (A), obtained according to the abovementioned protocol, are joined together with LOCTITE EDAG 423SS E&C carbon adhesive as spacer material so as to form a reservoir. This reservoir is filled with the following enzyme and mediator:
HRP enzyme: Peroxidase from horseradish; CAS 9003-99-0; Sigma Aldrich
Redox mediator (ABTS): 2,2′-azino-bis(3-ethylbenzothiazoline-6-sulfonic acid)diammonium salt; CAS 30931-67-0; Fluka

The HRP enzyme (2 mg) and the ABTS mediator (1 mg) are simply deposited in powder form during the manufacture of the electrode. A phosphate buffer at pH 7.4 is used during the operation of the electrode.

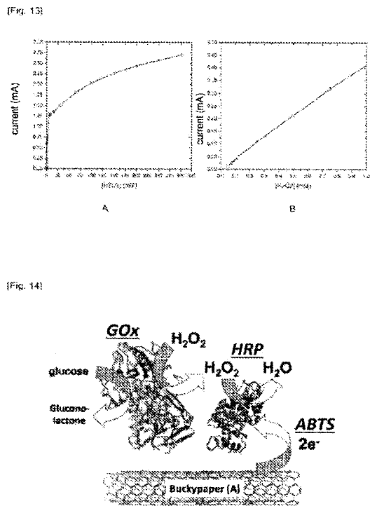

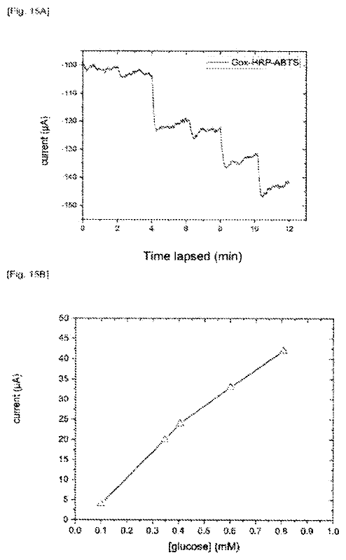

An argon chronoamperometric detection curve at −0.2 V vs Ag/AgCl in PB at pH 7.4 makes it possible to record the response of the catalytic current for reducing H₂O₂ by HRP as a function of the H₂O₂ concentration in solution. This curve is shown in FIGS. 13A and B (partial enlargement). The HRP in solution in the reservoir functions and the electron transfer via ABTS is ensured.

### Example 6: Bioelectrode with GOx-HRP-ABTS Bi-Enzymatic Cavity

An electrode is produced on the principle of the preceding examples. In order to produce a bi-enzyme electrode, two sheets of buckypaper (A), obtained according to the abovementioned protocol, are joined together with LOCTITE EDAG 423SS E&C carbon adhesive as spacer material so as to form a reservoir. This reservoir is filled with the following two enzymes and mediator:

Enzyme no. 1 (GOx): Glucose Oxidase from Aspergillus niger; CAS 9001-37-0; Sigma Aldrich.

Enzyme no. 2 (HRP): Peroxidase from horseradish; CAS 9003-99-0; Sigma Aldrich.

Redox mediator (ABTS): 2,2′-azino-bis(3-ethylbenzothiazoline-6-sulfonic acid)diammonium salt; CAS 30931-67-0; Fluka.

An electrode containing 1.5 mg of GOx, 1.5 mg of HRP and 1 mg of ABTS was produced. The enzymes and the mediator are simply deposited in powder form during the manufacture of the electrode. A phosphate buffer at pH 7.4 is used during the operation of the electrode. Glucose is injected into the aqueous buffer wherein the cavity electrode is plunged, it diffuses through the buckypapers and enters into the cavity where it is oxidized by the GOx enzyme.

GOx catalyzes the reduction of dioxygen O₂ in hydrogen peroxide H₂O₂ in the presence of glucose following the reaction:

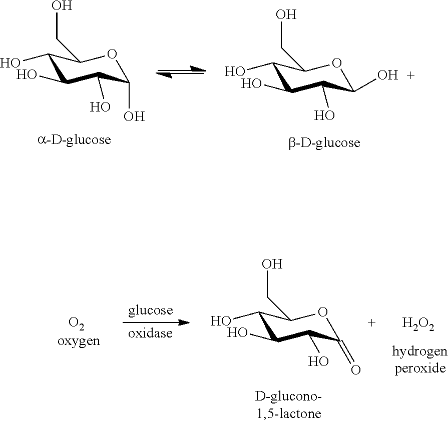

HRP: HRP reduces hydrogen peroxide H₂O₂ in water H₂O.

ABTS: ABTS is a redox mediator that makes it possible to transfer electrons from HRP to the buckypaper current collector (A)

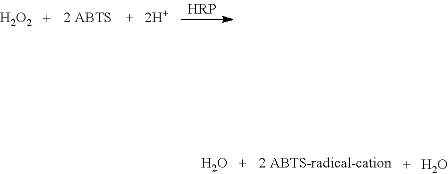 [SMILES: "H2O2" ; O ; O]

A chronoamperometric detection curve (ambient air) at 0 V vs Ag/AgCl in PB at pH 7.4 (FIG. 15A) makes it possible to record the response of the catalytic current for reducing H₂O₂ by HRP as a function of the glucose concentration in solution. Indirectly, this results in the first catalytic reaction of GOx.

With each glucose injection, a change in level of the current respond of HRP is observed. This establishes the successive reactions of the production of H₂O₂ by GOx (in the presence of O₂ dissolved in solution) and of the reduction of H₂O₂ by HRP; the electrical connection being ensured by ABTS.

The originality of this enzymatic cascade electrode as exemplified in Example 6 is its easy operation without a step of functionalizing the surfaces by specific mediators. The enzymes and the mediator are simply deposited in powder form during the manufacture of the electrode and can be considered as functioning in solution in the cavity.

### LIST OF DOCUMENTARY REFERENCES

1. P. Cinquin, C. Gondran, F. Giroud, S. Mazabrard, A. Pellissier, F. Boucher, J.-P. Alcaraz, K. Gorgy, F. Lenouvel, S. Mathé, P. Porcu, S. Cosnier; 2010 “A Glucose BioFuel Cell Implanted in Rats” PLOS ONE Vol. 5, Issue 5, (2010) e10476.
2. J. L. Hammond, A. J. Gross, F. Giroud, C. Travelet, R. Borsali, S. Cosnier. “Solubilized enzymatic fuel cell (SEFC) for quasi-continuous operation exploiting carbohydrate block copolymer glyconanoparticle mediators.” ACS Energy Lett., 4 (2019) 142-148. DOI: 10.1021/acsenergylett.8b01972.
3. Zehua Li, Zepeng Kang, Bo Wu, Zhiguang Zhu. “A MXene-based slurry bioanode with potential application in implantable enzymatic biofuel cells.” J. Power Sources 506 (2021) 230206.

What is claimed is:

1. An electrode comprising:
an electronically conductive material at least in part forming a reservoir, said material being permeable and porous;
an enzyme compound disposed in the reservoir; and
optionally, a current collector.

2. The electrode according to claim 1, wherein between 40 and 60% of the internal surface of said reservoir consists of said electronically conductive material.

3. The electrode according to claim 1, wherein said conductive material is hydrophilic.

4. The electrode according to claim 1, wherein said conductive material comprises carbon nanotubes and advantageously multi-walled carbon nanotubes.

5. The electrode according to claim 4, said carbon nanotubes having an average diameter ranging from 10 nm to 30 nm.

6. The electrode according to claim 1, wherein said conductive material is in the form of at least one sheet.

7. The electrode according to claim 1, wherein said reservoir comprises conductive material sheets bonded together by an adhesive.

8. The electrode according to claim 1, wherein the reservoir comprises an adhesive layer having a thickness, said thickness forming a part of the reservoir and define the height of the reservoir.

9. The electrode according to claim 1, wherein said reservoir has a total internal volume ranging from 10 μL to 500 μL.

10. The electrode according to claim 1, wherein said enzyme compound is in powder form.

11. The electrode according to claim 1, wherein said enzyme compound comprises two enzymes of which one is a GOx.

12. The electrode according to claim 1, further comprising at least one redox mediator.

13. The electrode according to claim 1, wherein the electronically conductive material is not functionalized by a redox mediator.

14. A biocell comprising at least one electrode as defined in claim 1.

15. (canceled)

16. The electrode according to claim 1, wherein said material is in the form of sheets having a thickness ranging from 50 to 500 μm.

17. The electrode according to claim 1, wherein said reservoir has a total internal volume ranging from 10 μL to 100 μL.

18. The electrode according to claim 1, wherein said reservoir has a total internal volume ranging from 10 μL to 50 μL.

19. The electrode according to claim 12, wherein the at least one redox mediator is chosen in the group consisting of 1,4-naphtoquinone, 9,10-phenanthrenequinone, 1,10-phenanthroline-5,6-dione, 9,10-anthraquinone, phenanthrene, 1,10-phenanthroline, 5-methyl-1,10-phenanthroline, pyrene, 1-aminopyrene, 1-pryenebutyric acid, ABTS, protoporphyrines IX, and mixtures of two or plus of these compounds.

20. The electrode according to claim 19, wherein the protoporphyrines IX are hemin.

21. The biocell of claim 14, wherein the biocell is a single-use and/or biodegradable biocell.

22. An implanted medical device comprising the biocell of claim 14.

23. The implanted medical device of claim 22, wherein the device is a cardiac stimulator, a pump, a sensor or a bionic implant.

24. The electrode according to claim 4, wherein said carbon nanotubes are multi-walled carbon nanotubes.
